(19) United States
(12) Patent Application Publication
(10) Pub. No.: US 2008/0219750 A1
(43) Pub. Date: Sep. 11, 2008

(54) Dual prep applicator

(76) Inventor: Marjorie Siegel, Mill Valley, CA (US)

Correspondence Address: JAMES RAY & ASSOCIATES, 2640 PITCAIRN ROAD, MONROEVILLE, PA, 15146, US

(21) Appl. No.: 12/074,422

(22) Filed: Mar. 4, 2008

Related U.S. Application Data
(60) Provisional application No. 60/892,933, filed on Mar. 5, 2007.

(51) Int. Cl. A61M 31/00; B05C 21/00

(52) U.S. Cl. 401/196; 604/2

(57) ABSTRACT

An apparatus for prepping surgery patients in an operating room. The apparatus comprises a shaft member having a predetermined shape and a predetermined size. An antiseptic solution is disposed in the shaft member, such antiseptic solution being present in a predetermined amount. A sponge member having a predetermined size is disposed on each end of the shaft member. There is a means disposed either in or on the shaft member for releasing the antiseptic solution to the sponge member.

### CROSS REFERENCE TO RELATED APPLICATIONS

This application is closely related to and claims benefit from U.S. Provisional Application Ser. No. 60/892,933 filed Mar. 5, 2007.

### FIELD OF THE INVENTION

The present invention relates, in general, to a medical applicator and, more particularly, the present invention relates to a double prepping applicator for use in prepping surgery patients in the operating room.

### BACKGROUND OF THE INVENTION

Applicators are presently used in prepping surgery patients in the operating room. The purpose of the applicator is to apply antiseptic solution to the patient=s body just before an incision is made. This is the standard practice known as “prepping”, the purpose of which is to reduce the risk of post-operative infection.

In the past, preps were done using pre-packaged, disposable kits containing gloves, gauze sponges and bowls to hold the prep solution, which was added at the time of use.

More recently, simplified prep kits were introduced. These kits consist of one item, an applicator, which is basically a sponge on a hollow stick, which contains the solution in the shaft.

By squeezing the shaft, the nurse cracks a glass vial, which releases the solution into the sponge. The prep is then painted onto the skin according to aseptic technique. Aseptic technique requires that prepping be done in a specific sequence, moving from the location of the incision outward, without going back. This is known as going from “clean” to “contaminated” areas. Once a sponge touches a contaminated, or less clean area, it may not be used on a “clean” area.

The desirable qualities of applicators include:
(1) allows the surgical nurse to apply the prep solution using the prep aseptic procedure;
(2) improves the efficiency of prepping by saving time; and
(3) decreases the cost of materials, including the cost of maintaining and storing inventory.

Sometimes in prepping it is necessary to open two or more prep applicator kits, even though there is enough solution in one kit, the reason is due to the requirements of aseptic technique. For instance, if a nurse misses an area the nurse may not use the applicator to go back, once it has moved to a “contaminated” area. There are also numerous instances when this routinely occurs due to the nature of the surgery, such as prepping for Total Knee Replacement or in many gynecological surgeries. An additional problem is that the sponge on the applicator is only one size. Sometimes the standard size is too small or too big for body contours, and so is inefficient or awkward to use.

Thus, it would be advantageous if there were a prep kit which would help in making the prep procedure more efficient and less wasteful, both in time and material.

### SUMMARY OF THE INVENTION

In a first aspect the present invention provides an apparatus for prepping surgery patients in an operating room. The apparatus comprises a shaft member having a predetermined shape and a predetermined size. An antiseptic solution is disposed in the shaft member, such antiseptic solution being present in a predetermined amount. A sponge member having a predetermined size is disposed on each end of the shaft member. There is a means that is disposed either in or on the shaft member for releasing the antiseptic solution to the sponge member.

In a second aspect, the present invention provides, in combination with an applicator for prepping surgery patients prior to surgery, having a sponge disposed on one end of a substantially hollow shaft member and having a predetermined amount of an antiseptic solution disposed within the shaft member closely adjacent to the sponge and a means for releasing the antiseptic solution to such sponge, the improvement comprises a sponge member having a predetermined size and a predetermined shape disposed on each end of the shaft member. There is a predetermined amount of antiseptic solution disposed within each end of the shaft member closely adjacent each sponge member and a means that is either disposed in or on the shaft member for releasing the antiseptic solution to the sponge member.

### OBJECTS OF THE INVENTION

It is, therefore, one of the primary objects of the present invention to provide a dual headed applicator which has a sponge on each end of the shaft.

Another object of the present invention is to provide an applicator which would have different size sponges on each end of the applicator.

Still another object of the present invention is to provide an applicator which has a large sponge on one end and a smaller sponge on the opposite end so as to better fit the bodies contour.

Yet another object of the present invention is to provide an applicator that is modified so as to deliver solution to both ends.

Another object of the present invention is to provide an applicator in which the shaft would be divided into two separate vials.

In addition to the various objects and advantages of the invention which have been described in some specific detail above it should be noted that various other objects and advantages of the present invention will become more readily apparent to those persons who are skilled in the relevant art from the following more detailed description, particularly when such description is taken in conjunction with the appended claims.

### BRIEF DESCRIPTION OF THE DRAWINGS

FIG. 1 is a side perspective view of a prior art prep applicator.

FIG. 2 is a side perspective view of an applicator according to an embodiment of the present invention.

FIG. 3 is a top perspective view of a different prior art prep applicator.

FIG. 4 is a top perspective view of the prep applicator according to an alternate embodiment of the invention.

FIG. 5 is a top perspective view of yet a different prior art prep applicator.

FIG. 6 is a top perspective view of the prep applicator according to an alternate embodiment of the invention.

FIG. 7 is a side perspective view of still another prior art prep applicator.

FIG. 8 is a side perspective view of an applicator according to another embodiment of the present invention.

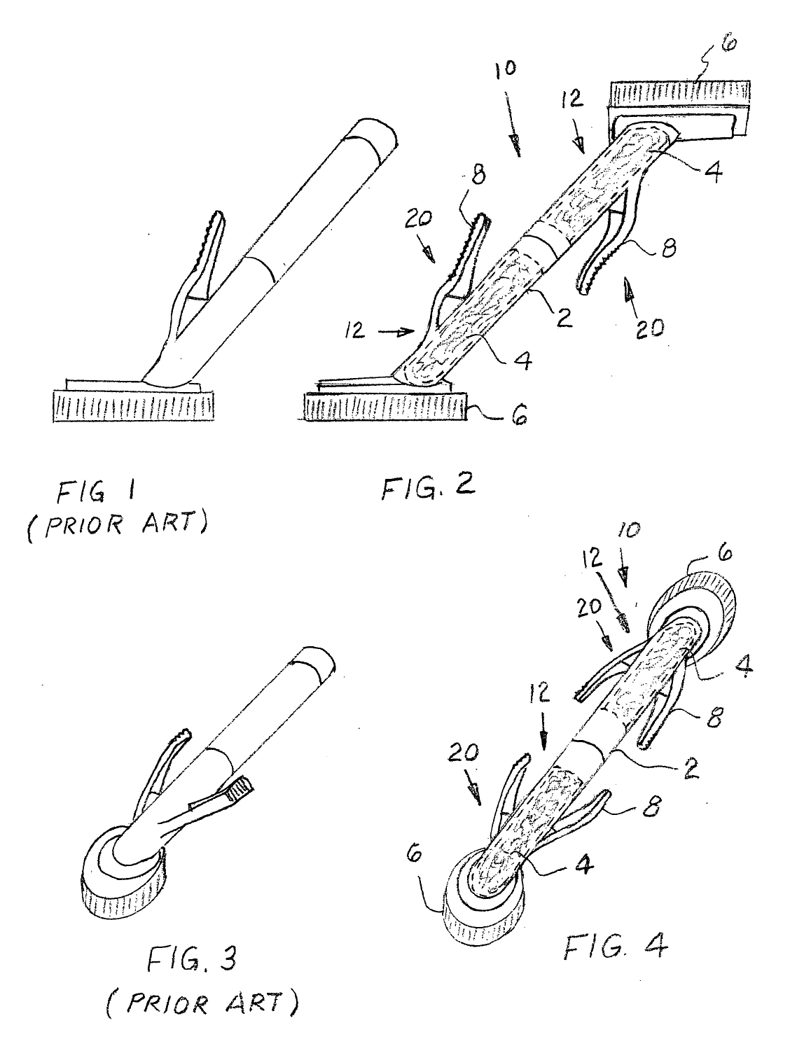

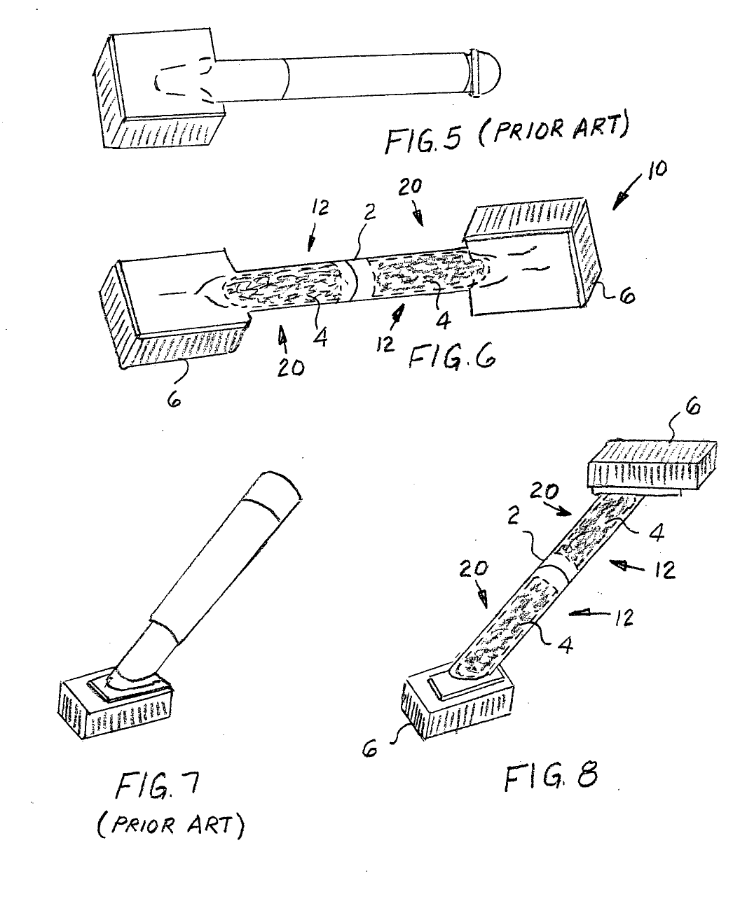

### BRIEF DESCRIPTION OF THE PRESENTLY PREFERRED AND VARIOUS ALTERNATIVE EMBODIMENTS OF THE INVENTION

Prior to proceeding with the more detailed description of the present invention it should be noted that, for the sake of clarity, identical components which have identical functions have been designated by identical reference numerals throughout the several views illustrated in the drawings.

In a first aspect the present invention provides an apparatus, generally designated 10, for prepping surgery patients in an operating room. The apparatus 10 comprises a shaft member 2 having a predetermined shape and a predetermined size. An antiseptic solution 4 is disposed in the shaft member 2, such antiseptic solution 4 being present in a predetermined amount. A sponge member 6 having each of a predetermined size and a predetermined shape is disposed on each end of the shaft member 2.

There is a means 20 disposed one of in and on the shaft member 2 for releasing the antiseptic solution 4 to the sponge member 6.

The antiseptic solution 4 is disposed in a container member disposed within the shaft member 2. Such container member is a vial. The container member is easily fractured so as to release the antiseptic solution 4.

Such shaft member 2 further includes at least one projection 8 that is disposed on an outer surface thereof. Such projection 8 is closely adjacent the sponge member and extends outwardly therefrom. The antiseptic solution 4 is released by one of squeezing the tube shaped shaft member 2 or pressing on the projection 8 thereby squeezing such container member 12 disposed in shaft member 2 and providing antiseptic solution 4 to the sponge member 6.

The predetermined shape of the shaft member is selected from one of a tube, a rectangle, an octagon and a hexagon. It is presently preferred that such shaft member has a tube shape.

The present invention provides that such antiseptic solution 4 is disposed in two container members 12 since there are two sponge members 6. Thus, there is a separate container member 12 for each sponge member 6.

The predetermined shape of such sponge member 6 is one of circular, square and rectangular. When the predetermined shape of such sponge member is square such predetermined size can be one of two different sizes. The at least one sponge member is either between about 1¾ inches and about 2⅛ inches on each side as a large sponge or it can be a smaller sponge that is between about 1⅛ inches and about 1½ inches on each side. It is presently preferred that such larger sponge be substantially 2 inches on each side while it is preferred that the smaller sponge be substantially 1⅜ inches on each side. It is within the concept of the invention that such sponge 2 on each end of the shaft member 2 could be the same size or a different size. It is presently preferred that the applicator have a different size sponge 6 on each end of the shaft member 2. Thus, one end of the shaft member 2 could have the larger sponge as described supra while the other end of the shaft member 2 could have a smaller sponge disposed thereon. In this way the different sizes would also permit a choice for the nurse as to which would be most suitable for the body contour near the surgery.

In the case where the predetermined shape is circular the predetermined size can have a diameter of between about 1⅛ and about 1½ inches as a smaller sponge or between about 1¾ and about 2⅛ inches for a larger sponge.

The sponge member 6 can be disposed on an end of the shaft member 2 in one of an angle or in a straight line. It is presently preferred that each such sponge member 6 on each end of the shaft member be disposed at an angle.

In a second aspect the present invention provides in combination with an applicator for prepping surgery patients prior to surgery having a sponge 6 disposed on one end of a substantially hollow shaft member 2 and having a predetermined amount of an antiseptic solution 4 disposed within the shaft member 2 closely adjacent the sponge 6 and a means 20 for releasing the antiseptic solution 4 to the sponge 6, the improvement comprises a sponge member 6 having each of a predetermined size and a predetermined shape disposed on each end of shaft member 2. There is a predetermined amount of an antiseptic solution 4 disposed within each end of the shaft member 2 closely adjacent each sponge member 6 and a means 20 that is disposed one of in and on each end of the shaft member 2 for releasing the antiseptic solution 4 to each sponge member 6.

It is presently preferred that such antiseptic solution 4 is disposed in two separate container members that are disposed within the shaft member 2 closely adjacent each end thereof.

Such shaft member 2 further includes at least one projection 8 disposed on an outer surface thereof closely adjacent a sponge member 6 and extending outwardly therefrom. It is presently preferred that the shaft member 2 includes two projections 8, each one of the two projections 8 is disposed on an outer surface of each end of the shaft member 2, each projection 8 is closely adjacent a sponge member 6 and extends outwardly therefrom.

Thus, the present invention provides a double prep applicator for use in prepping surgery patients in the operating room. The prepping of surgery patients is designed to reduce the risk of post operative infection. Antiseptic solution is applied to the patients body surface just before the incision is made. As stated previously, the process needs to be performed according to predetermined requirements, which specify that the prepping must be done in a specific sequence, moving in the location of the incision outwardly without going back. This is known as going from “clean” to “contaminated” areas. Once a sponge touches a contaminated, or less clean area, it may not be used on a “clean” area. That means that if a spot is missed that nurse cannot use the applicator to go back to a place that is nearer the incision after going further away. In this case a new, unused applicator is needed for the missed areas.

The dual prep applicator is similar to prior art single sponge applicators and feature a tube shaped solution container (shaft member) that is squeezed to release the antiseptic solution onto the sponge tip. The present invention provides a sponge disposed on each end of the shaft member. Each sponge has its own container with antiseptic solution. This eliminates the need to open an entirely new applicator when the applicator has moved to a contaminated area. The present invention is capable of providing antiseptic solution to each end of the applicator. This can be done by providing an enlarged area with more antiseptic solution or, alternatively, dividing the solution into two separate containers, one container for each end of the applicator.

It is within the scope of the invention that the sponges on each end of the applicator could be the same or different sizes. It is presently preferred that the sponges be different sizes. One of the sponges would be a standard size sponge, as is currently in use, which would be between about 1¾ inches and about 2⅛ inches, while the other sponge could a smaller version being between about 1⅛ inches and about 1½ inches. Such sponges could also have a circular shape. The two different sizes would also be useful for the different body contours of different patients since sometimes the standard size is too big for some body contours and, thus, is inefficient and awkward to use.

While a presently preferred embodiment and alternate embodiments of the present invention have been described in detail above, it should be understood that various other adaptations and/or modifications of the invention can be made by those persons who are particularly skilled in the art without departing from either the spirit of the invention or the scope of the appended claims.

I claim:

1. An apparatus for prepping surgery patients in an operating room, said apparatus comprises:
(a) a shaft member having each of a predetermined shape and a predetermined size;
(b) an antiseptic solution disposed in said shaft member, said antiseptic solution being present in a predetermined amount;
(c) a sponge member having a predetermined size disposed on each end of said shaft member; and
(d) a means for releasing said antiseptic solution to said sponge member is disposed one of in and on said shaft member.

2. The apparatus, according to claim 1, wherein said antiseptic solution is disposed in a container member disposed within said shaft member.

3. The apparatus, according to claim 2, wherein said shaft member further includes at least one projection disposed on an outer surface thereof closely adjacent said sponge member and extending outwardly therefrom.

4. The apparatus, according to claim 3, wherein said antiseptic solution is released by one of squeezing said shaft member and pressing on said projection thereby squeezing said container member disposed in said shaft member and providing antiseptic solution to said sponge member.

5. The apparatus, according to claim 1, wherein said predetermined shape of said shaft member is selected from one of a tube, a rectangle, an octagon and a hexagon.

6. The apparatus, according to claim 6, wherein said predetermined shape is a tube.

7. The apparatus, according to claim 2, wherein said antiseptic solution is disposed in two container members.

8. The apparatus, according to claim 7, wherein there is a separate container member for each sponge.

9. The apparatus, according to claim 1, wherein said predetermined shape of said sponge member is one of circular, square and rectangular.

10. The apparatus, according to claim 10, wherein said predetermined shape is square.

11. The apparatus, according to claim 10, wherein said predetermined size of at least one said sponge member is one of being between about 1¾ inches and about 2⅛ inches on each side for a larger said sponge and between about 1⅛ inches and about 1½ inches on each side for a smaller said sponge.

12. The apparatus, according to claim 9, wherein said predetermined shape is circular.

13. The apparatus, according to claim 12, wherein said sponge member has a predetermined diameter of between about 1⅛ and about 1½ inches for a smaller size said sponge.

14. The apparatus, according to claim 1, wherein said sponge member is disposed on an end of said shaft member in one of an angle and in a straight line.

15. The apparatus, according to claim 15, wherein said sponge member is disposed on an end of said shaft member at an angle.

16. The apparatus, according to claim 1, wherein said predetermined size of each said sponge member disposed on each end of said shaft member is different.

17. In combination with an applicator for prepping surgery patients prior to surgery having a sponge disposed on one end of a substantially hollow shaft member and having a predetermined amount of an antiseptic solution disposed within said shaft member closely adjacent said sponge and a means for releasing said antiseptic solution to said sponge, the improvement comprising:
(a) a sponge member having a predetermined size and a predetermined shape disposed on each end of said shaft member;
(b) a predetermined amount of antiseptic solution disposed within each end of said shaft member closely adjacent each said sponge member; and
(c) a means disposed one of in and on said shaft member for releasing said antiseptic solution to said sponge member.

18. The applicator, according to claim 18, wherein said antiseptic solution is disposed in two separate container members that are disposed within said shaft member closely adjacent each end thereof.

19. The apparatus, according to claim 18, wherein said shaft member further includes at least one projection disposed on an outer surface thereof closely adjacent said sponge member and extending outwardly therefrom.

20. The apparatus, according to claim 19, wherein said shaft member includes two projections, each one of said two projections disposed on an outer surface of each end of said shaft member, said each projection closely adjacent said sponge member and extending outwardly therefrom.
